(19) United States
(12) Patent Application Publication
(10) Pub. No.: US 2011/0245688 A1
(43) Pub. Date: Oct. 6, 2011

(54) SYSTEM AND METHOD OF PERFORMING ELECTROCARDIOGRAPHY WITH MOTION DETECTION

(75) Inventors: Dhiraj Arora, Niskayuna, NY (US); Harry Kirk Mathews, JR., Clifton Park, NY (US)

(73) Assignee: GENERAL ELECTRIC COMPANY, Schenectady, NY (US)

(21) Appl. No.: 12/751,787

(22) Filed: Mar. 31, 2010

(51) Int. Cl. A61B 5/0205; A61B 5/11

(52) U.S. Cl. 600/483; 600/595

(57) ABSTRACT

A system in accordance with present embodiments includes an electrocardiograph, a plurality of sensors communicatively coupled with the electrocardiograph, wherein each of the plurality of sensors comprises an electrode capable of detecting electrical impulses generated by a patient's body and transmitting signals indicative of detected electrical impulses to the electrocardiograph. In one embodiment, the system also includes a motion detection feature communicatively coupled with the electrocardiograph, wherein the motion detection feature is capable of detecting movement of the patient's body and providing signals indicative of detected movement to the electrocardiograph, and wherein the electrocardiograph is capable of detecting a particular type of patient motion and/or patient position based on the signals indicative of the detected motion, capable of providing output based on the signals indicative of the detected electrical impulses, and capable of providing output based on the signals indicative of the detected movement.

### BACKGROUND OF THE INVENTION

The subject matter disclosed herein relates to electrocardiography. More specifically, present embodiments are directed to a system and method for addressing issues related to false alarms obtained during the performance of electrocardiography and supplementing the information obtained via electrocardiography with motion data.

Electrocardiography is a diagnostic procedure performed by a device called an electrocardiograph, wherein a patient's heart activity is recorded electronically by measuring electrical impulses generated by the heart as it is beating. Electrical impulses begin in the sinoatrial node of the heart and travel through a network of nerve pathways around the heart muscle. The impulses cause the heart muscle to contract, inducing systole, by stimulating muscle fibers. Different areas of the heart may experience different levels of electrical activity. This electrical activity can be detected through the patient's skin. Accordingly, an electrocardiograph includes electrodes that are placed on the patient's skin in different positions relative to the heart such that each electrode measures electrical activity in a different part of the heart. Electrodes are traditionally placed in specific areas near the heart and on the patient's limbs. The product of the performance of electrocardiography is typically an electrocardiogram (ECG), which is a graphical record of the cardiac cycle produced by the electrocardiograph. The ECG may include measurements of the voltage between the electrodes and the muscle activity from the different areas of the heart based on the various placements of the electrodes.

Electrocardiographs and the resulting ECG are often utilized to measure and diagnose arrhythmia or abnormal heart rhythms, weakness in different areas of the heart, damage to conductive tissue, imbalances in electrolytes, and so forth. Additionally, electrocardiographs are often utilized to continuously monitor patients in hospitals, clinics, and so forth. During patient monitoring, if an ECG indicates certain patient conditions are present, an alarm may be generated to notify healthcare providers of the condition. However, due to noise in the ECG signal, various false alarms may be generated. Such false alarms can become a nuisance and may cause inefficiencies in patient care.

### BRIEF DESCRIPTION OF THE INVENTION

Certain embodiments commensurate in scope with the originally claimed invention are summarized below. These embodiments are not intended to limit the scope of the claimed invention, but rather these embodiments are intended only to provide a brief summary of possible forms of the invention. Indeed, the invention may encompass a variety of forms that may be similar to or different from the embodiments set forth below.

In one embodiment, a system includes an electrocardiograph, a plurality of sensors communicatively coupled with the electrocardiograph, wherein each of the plurality of sensors comprises an electrode capable of detecting electrical impulses generated by a patient's body and transmitting signals indicative of detected electrical impulses to the electrocardiograph. In one embodiment, the system also includes a motion detection feature communicatively coupled with the electrocardiograph, wherein the motion detection feature is capable of detecting movement of the patient's body and providing signals indicative of detected movement to the electrocardiograph, and wherein the electrocardiograph is capable of detecting a particular type of patient motion and/or patient position based on the signals indicative of the detected motion, capable of providing output based on the signals indicative of the detected electrical impulses, and capable of providing output based on the signals indicative of the detected movement.

In one embodiment, a electrocardiograph monitor comprises one or more inputs capable of receiving signals from an electrode capable of detecting electrical impulses from a patient's body and signals from a motion detection feature capable of detecting movement of the patient's body, a processor capable of identifying a type of motion and/or posture of the patient's body based on the signals from the motion detection feature, and an alarm mechanism capable of providing an audible, tactile, or visual alert upon detection of a certain level or pattern of the electrical impulses and capable of providing a corresponding indication of the type of motion and/or posture of the patient's body based on the signals from the motion detection feature.

In one embodiment, a method includes receiving measurements from a motion detection feature capable of detecting patient movement and receiving measurements from an electrode capable of detecting electrical impulses from a patient at a electrocardiograph. In one embodiment, the method also includes recording the measurements from the motion detection feature and the electrode, identifying the presence of a level or type of patient movement based on the recorded measurements, and suppressing an alarm generated by the measurements from the electrode based on the identified movement.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an embodiment of an electrocardiograph including sensors coupled to a patient;

FIG. 2 depicts a perspective view of one embodiment of a sensor with a coupling feature, an electrode, and an accelerometer integral with one another;

FIG. 3 depicts a perspective view of one embodiment of an accelerometer sensor including an accelerometer that may be utilized separate from an electrode sensor;

FIG. 4 illustrates one embodiment of an ECG and one embodiment of an accelerometer graph obtained essentially simultaneously during a patient's scratching motion;

FIG. 5 illustrates one embodiment of an ECG and one embodiment of an accelerometer graph obtained essentially simultaneously during a patient's coughing motion;

FIG. 6 illustrates one embodiment of an ECG and one embodiment of accelerometer graph obtained essentially simultaneously during a time period wherein a patient changed position from supine to sitting and back to supine; and

FIG. 7 illustrates one embodiment of a process or algorithm that may be performed by a system in accordance with present embodiments, wherein certain motion types are identified and actions are taken to limit false alarms or to facilitate identification of potential causes of false alarms.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements.

It is now recognized that motion artifacts often causes false alarms during electrocardiograph monitoring. For example, it is now recognized that certain repetitive motion artifacts often mimic pathological arrhythmias resulting in false alarms. An excessive number of false alarms generally make it difficult for medical attendants to address each alarm. Indeed, the more alarms that are presented, the more time attendants have to spend addressing the alarms. This can be inefficient and expensive. Indeed, such false alarms can be particularly inconvenient for hospitals and clinics that are attempting to decrease a number of medical attendants per patient.

Accordingly, present embodiments are directed to detecting and measuring patient motion with accelerometers or other motion detection devices (e.g., a gyro or optical devices) in order to address certain issues regarding false alarms based on motion artifact. Indeed, the motion measurements obtained via the accelerometers may be used to suppress alarms due to patient motion, provide additional information to an attendant, and/or compensate electrocardiograph signals to remove motion artifact. Specifically, for example, identified motion artifact may be utilized to suppress certain alarms that would otherwise be activated within a certain time period relative to the detected motion artifact. In another example, motion artifact may be identified and automatically eliminated from an electrocardiograph signal. As yet another example, indicators of detected motion artifact may be presented to medical attendants such that the reason for a particular false alarm quickly becomes clear. Additionally, it is presently recognized that certain motion detected by the accelerometers may also be utilized to provide additional metrics to facilitate analysis of the patient's condition. For example, under some conditions, the accelerometers may provide diagnostic information regarding respiration, cardiac heart rate, and so forth based on particular movement patterns. Indeed, changes in one or more accelerometer signals may be used to detect organ motion, cardiac, and lung activity. Further, changes in one or more accelerometer signals may be utilized to initiate a response (e.g., suppress alarms) based on the identification of certain types of motion. It should be noted that accelerometers are specifically discussed as features for detecting motion in the following examples. However, in some embodiments, different motion detection features may be utilized, such as self-contained gyros and optical features that may externally detect motion.

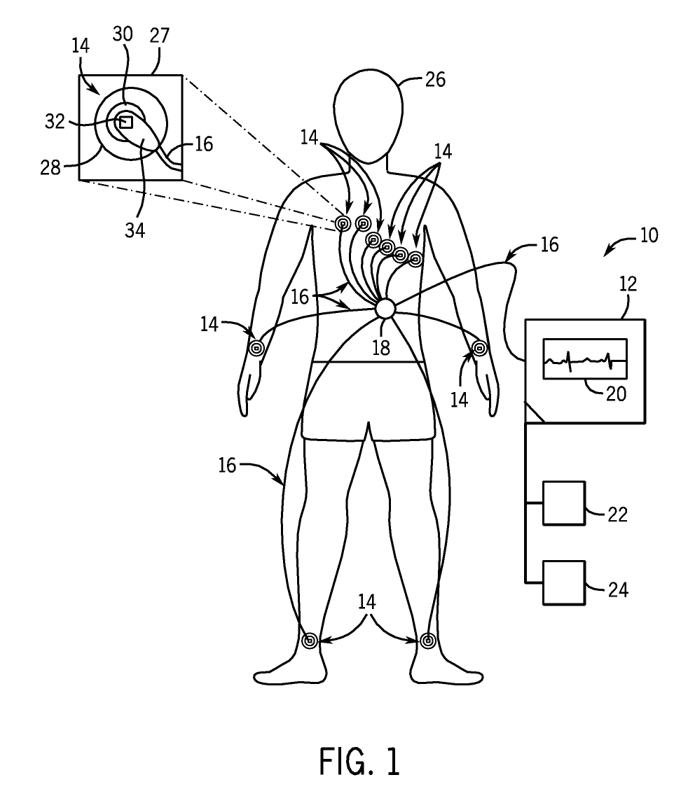

FIG. 1 illustrates an electrocardiograph 10 in accordance with present embodiments. Specifically, the electrocardiograph 10 includes a monitor 12, sensors 14, communication cables 16, a cable junction 18, a display 20, a processor 22, and a memory 24. In the illustrated embodiment, the sensors 14 are coupled to different areas on a patient 26. This coupling between the patient 26 and the sensors 14 may be achieved by an adhesive portion (e.g., a tacky base layer) of the sensor 14 or the like. The sensors 14 are coupled to the cable junction 18 via the individual communication cables 16 and the cable junction 18 couples with the monitor 12 via a single one of the communication cables 16. In other embodiments, different arrangements may be made. For example, each sensor 14 may directly communicate with the monitor 12. In some embodiments, each sensor 14 may communicatively couple with the monitor 12 wirelessly or couple with the cable junction 18, which may wirelessly communicate with the monitor 12. Additionally, in some embodiments a different number or placement of the sensors 14 may be utilized. In one embodiment, as will be discussed below, separate electrode and accelerometer sensors may be utilized. Such sensors may couple with a single input to the monitor 12 or the monitor 12 may include separate inputs for each sensor and/or each type of sensor.

In accordance with present embodiments, as more clearly illustrated in expanded view 27, each sensor 14 illustrated in FIG. 1 includes a coupling feature 28 (e.g., a thick tape piece with adhesive on one side) for attaching the sensor 14 to the patient 26, an electrode 30 for measuring electrical activity, and an accelerometer 32 for detecting motion. One or both of the electrode 30 and the accelerometer 32 may be integrated with a base 34 of the sensor 14 that communicatively couples with the communication cable 16. It should be noted that in some embodiments the accelerometer 32 may be replaced by a different motion detecting device, such as a gyro.

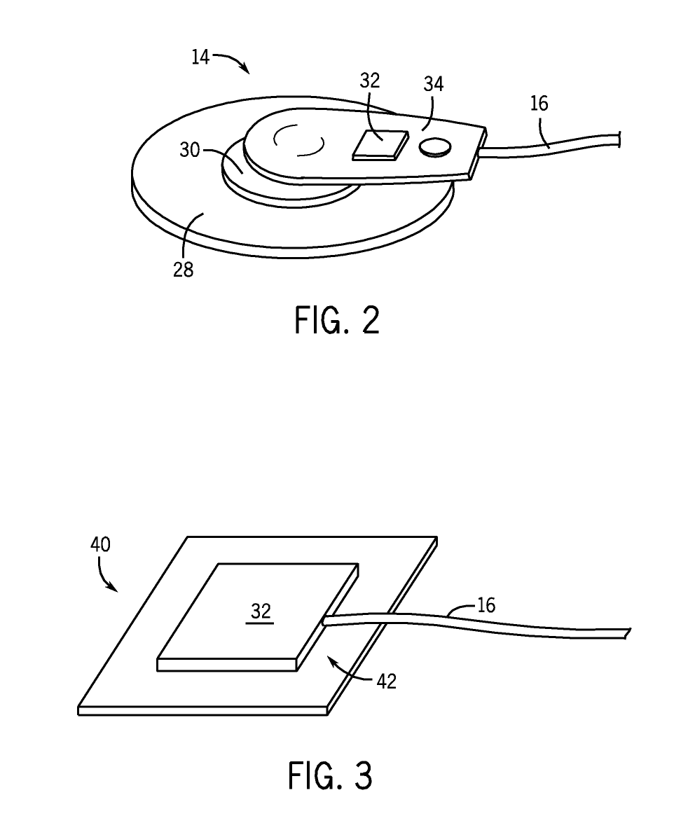

In the embodiment illustrated by FIG. 1, the accelerometers 32 are integral with the sensors 14. For example, FIG. 2 depicts a perspective view of one of the sensors 14 with the coupling feature 28, the electrode 30, and the accelerometer 32 integral with one another. However, in some embodiments, as illustrated in FIG. 3, the accelerometer 32 may be separate from the electrode 30. Indeed, FIG. 3 depicts a separate accelerometer sensor 40 that includes a coupling feature 42 (e.g., adhesive tape) and the accelerometer 32 coupled with one of the communication cables 16. The communication cable 16 illustrated in FIG. 3 may couple with the cable junction 18 along with other communication cables 16 from various types of sensors, or directly couple to a separate port of the monitor 12. One or more of the accelerometer sensors 40 may be separately applied to the patient 26 near traditional electrocardiograph sensors in accordance with present embodiments so that motion relative to one or more of the electrocardiograph sensors may be specifically identified. Further, in other embodiments, the accelerometer sensors 40 may be placed in different locations relative to the traditional electrocardiograph sensors or the sensors 14 to identify different types of motion

Present embodiments are generally directed to a process including simultaneously measuring and recording an ECG along with measuring and recording at least one accelerometer measurement. Changes in accelerometer measurements may be trended and correlated to detect patient motion (e.g., limb motion or organ motion). Indeed, the accelerometer measurement may be utilized to identify numerous different patient activities, such as a change of body position (e.g., from laying to sitting), coughing, skin scratching, and so forth. This information may be utilized for various diagnostic purposes. For example, it may be useful for a doctor examining an ECG to be made aware that a patient moved in a certain way during a certain period of time corresponding to data on the ECG. As indicated above, it is now recognized that patient motion such as this can cause motion artifact that initiates false alarms. Accordingly, a caregiver may be made aware of motion that potentially caused a false alarm. Further, upon detection of certain levels or types of patient motion, present embodiments may function to suppress the ECG signal, suppress alarms associated with the ECG signal, identify and provide notice of certain types of motion, and/or modify the ECG signal to eliminate and/or reduce the incidences of false alarms.

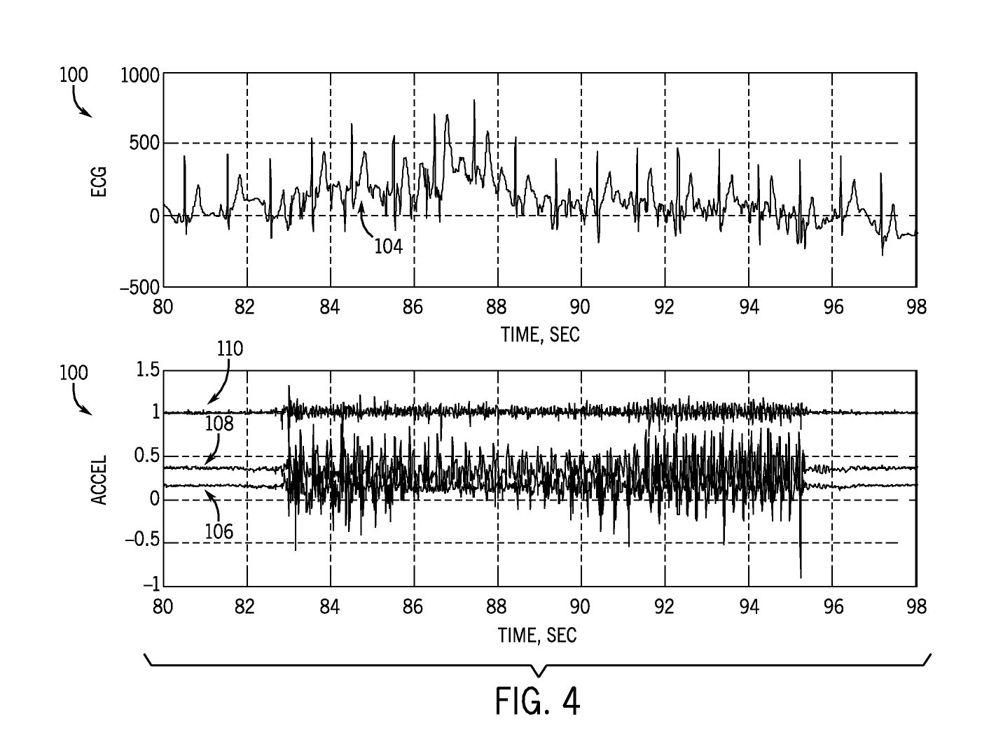

FIG. 4 includes an ECG 100 and an accelerometer graph 102 obtained during a patient's scratching motion. The ECG 100 includes a traditional ECG plot 104 and the accelerometer graph 102 includes measurements from a tri-axis accelerometer, which is an accelerometer that measures acceleration along three different axes. Thus, the accelerometer graph 102 includes data for each of the three directions, as represented by plot 106 (X-axis), plot 108 (Y-axis), and plot 110 (Z-axis). Both the ECG 100 and the accelerometer plot 102 were obtained from the same patient over the same time period. During the time these measurements were being taken, using his left arm, the patient scratched his skin near the top right electrode in a traditional electrocardiography arrangement of electrodes. It is believed that electrolytes activated by movement of the patient's arm along with noise created by movement of the sensor caused distortion in the ECG plot 104. Indeed, there is additional noise and a noticeable change in the baseline of the ECG plot 104 at approximately 83 seconds, which is near the time the scratching motion was initiated, as is clear from the corresponding measurements illustrated by the accelerometer graph 102. While the scratching continued until approximately the 95 second mark, the baseline of the ECG plot 104 appears to begin settling around the 90 second mark. This is believed to be due to stabilization of electrolytes after the initial arm movement and during the following finger movements, which are relatively small movements compared to adjusting the arm.

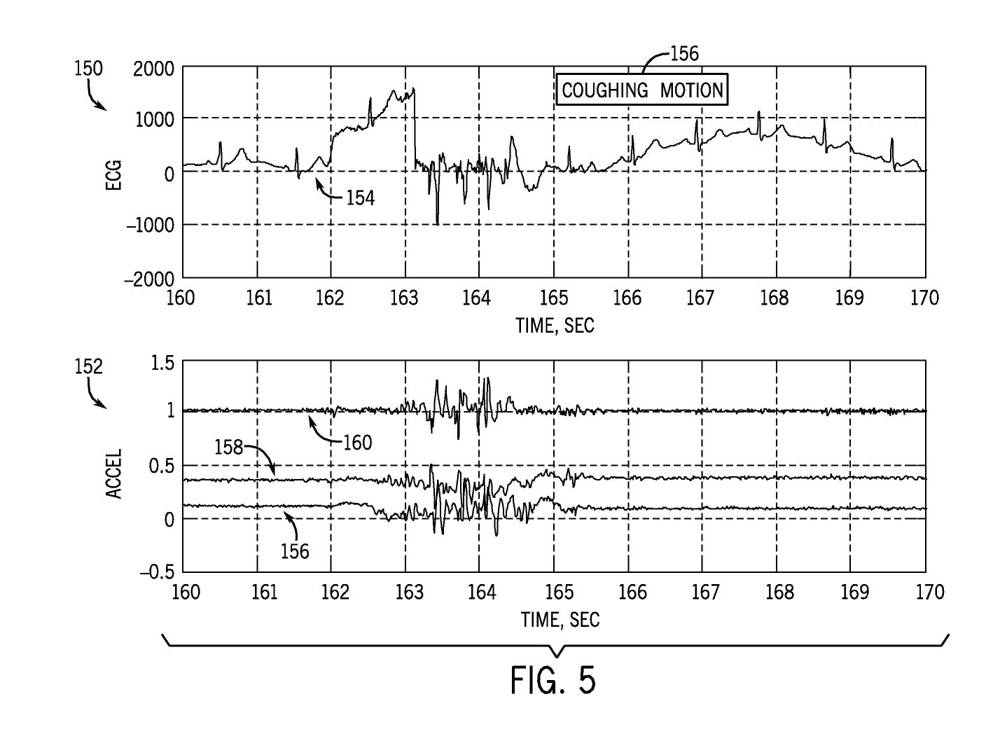

FIG. 5 includes an ECG 150 and an accelerometer graph 152 obtained during a patient's coughing motion. Like the ECG 100 and the accelerometer graph 102 in FIG. 4, the ECG 150 includes a traditional ECG plot 154, and the accelerometer graph 152 includes measurements from a tri-axis accelerometer. The accelerometer graph 152 includes data for each of the three directions, as represented by plot 156 (X-axis), plot 158 (Y-axis), and plot 160 (Z-axis). Both the ECG 150 and the accelerometer plot 152 were obtained from the same patient over the same time period. During the time these measurements were being taken, the patient inhaled and coughed. The occurrence of the cough can clearly be identified in the ECG 150 and the accelerometer graph 152. Indeed, there is a low frequency change in each plot of the accelerometer graph 152 at around 162 seconds, which is particularly clear in the plot 156. Such low frequency changes are statistically very significant relative to accelerometer noise. Also, at around 162 seconds, a large change in the ECG plot 154 begins. These changes in the ECG 150 and the accelerometer graph 152 occurred as a result of the patient inhaling. The following exhaling portion of the cough began around the 163 second mark and is indicated by significant disruption in both the ECG plot 154 and each plot in the accelerometer graph 152. While the accelerometer graph 152 substantially stabilizes shortly after the cough, the ECG plot 154 remains distorted for a period. Such time periods may be noted and accounted for in accordance with present embodiments. For example, alarms may be suppressed for a time period based on such empirical data after detecting the end of a coughing motion.

Changes in the ECG plots 104 and 150, such as those caused by the scratching motion and the cough, may correlate to certain alarm conditions. For example, the pattern created by the ECG plot 104 during the scratching motion may closely resemble an arrhythmia and a traditional electrocardiograph may emit an alarm upon receiving such measurements. However, present embodiments may suppress or delay such alarms based on the detected motion in the accelerometer graph 102 or provide an indication to a caregiver that such an alarm can be quickly dismissed. For example, upon detection of the scratching motion, an alarm may be suppressed or delayed for a period of time (e.g., a number of seconds after the last detected motion). If the motion goes away for a period of time and an alarm condition is still present, the alarm may be activated. In another example, a display may indicate the type of motion that occurred during the time the alarm was initiated so a caregiver can quickly identify the reason for the alarm. For example, a caregiver may review the ECG plot 154 or an automatic graphical indicator 156 of the type of motion along with the accelerometer graph 102 and discern that an alarm can be dismissed because it is merely due to a coughing motion. Indeed, even if the alarm is not silenced, present embodiments may improve a caregiver's efficiency by providing the caregiver with data related to potential false alarms generated by motion artifact. An indication of motion may include raw data of the motion (plots 156, 158, and 160) obtained from an accelerometer or explicitly identify certain types of motion (graphical indicator 156).

In some embodiments, all electrocardiography-related alarms may be suppressed when certain types of motion are detected. In other embodiments, based on empirical data, alarms that correspond to ECG plot trends likely to be confused with a pattern produced by a particular series of identified movements may be suppressed. For example, present embodiments may distinguish between a scratching motion and a coughing motion (based on empirical data obtained via clinical trials) and suppress different alarms depending on which type of motion was identified. Specifically, for example, a particular type of motion (e.g., scratching) may be known to mimic a particular alarm condition (e.g., arrhythmia) and alarms related to such alarm conditions may be suppressed for a period of time after last detecting the motion. Further, in some embodiments, a correlation may be made based on empirical data associating the detected motion with the particular type of distortion in the ECG plot 104, and the distortion due to the motion may be removed from the ECG plot. Such modification of the ECG plot 104 using empirical data may be useful for facilitating improved diagnosis during patient activity.

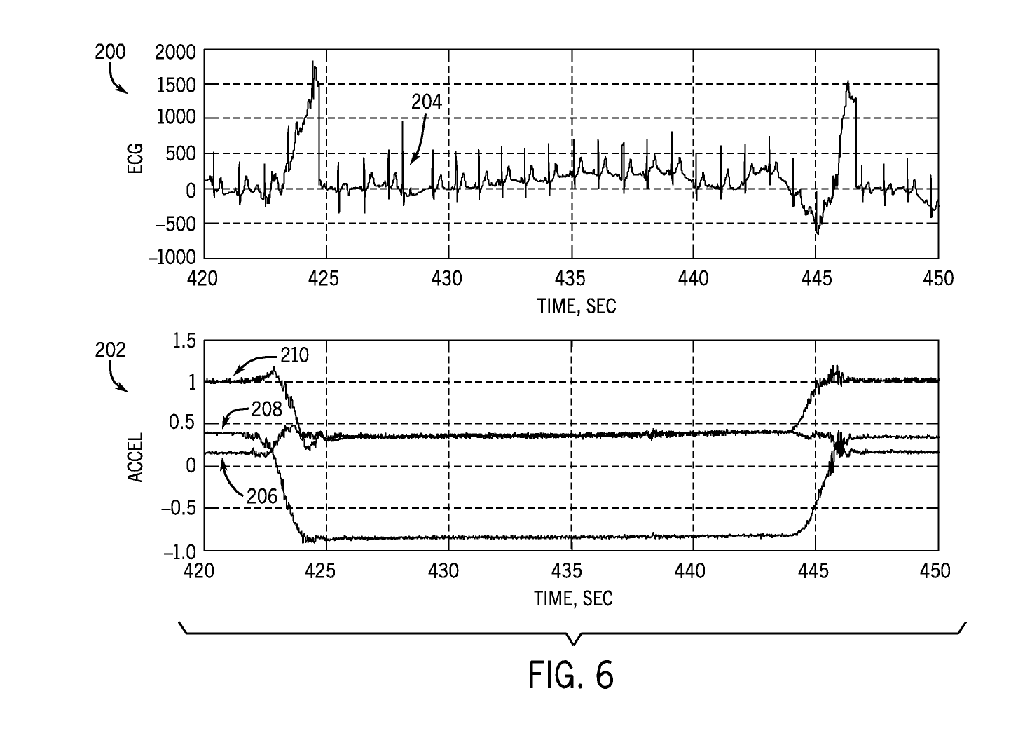

FIG. 6 includes an ECG 200 and an accelerometer graph 202 obtained during a time period wherein a patient changed position from a supine position to a sitting position and back to the supine position. This is an example of information that may be utilized by a physician when reviewing historical trend data. Indeed, the position of the patient during a certain time period may be useful to analyze the ECG 200 or other information. Like the ECG 100 and the accelerometer graph 102 in FIG. 4, the ECG 200 includes a traditional ECG plot 204, and the accelerometer graph 202 includes measurements from a tri-axis accelerometer. The accelerometer graph 202 includes data for each of the three directions, as represented by plot 206 (X-axis), plot 208 (Y-axis), and plot 210 (Z-axis). Based on the particular type of motion made by the patient during the acquisition of the information in the accelerometer graph 202, the three different axes of the tri-axis accelerometer clearly represent different changes. For example, plots 208 and 210 changed substantially when the patient moved from the lying position to sitting up at around 422 seconds because the accelerometer was positioned on the patient's chest and moved substantially in the Y and Z directions when the patient transitioned form supine to sitting. However, the plot 206 changed very little during this transitional movement because the patient did not move much in the X direction, as would be expected from a transition between supine and sitting. There was also a noticeable change in the ECG plot 204 during the movement of the patient. For example, there are large disturbances in the ECG plot 204 at approximately 223 seconds and 445 seconds, which are near the initiation of transition between the two positions. The noise in the ECG plot 204 may be correlated to the motion pattern provided by the various plots of the accelerometer graph 202 and utilized to reduce alarms and/or provide additional information to a caregiver.

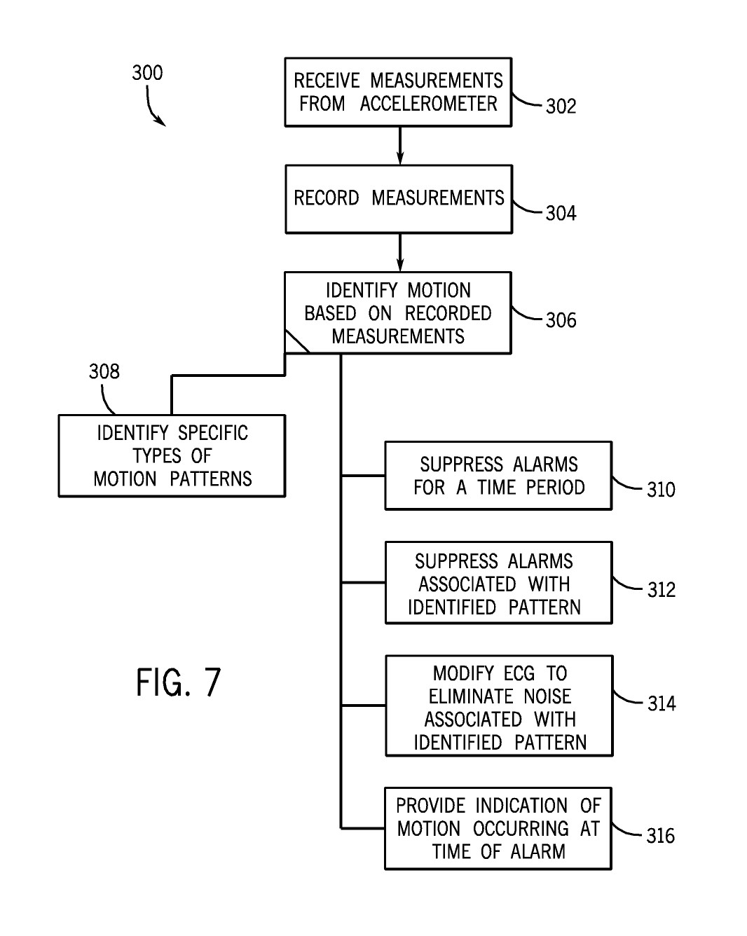

FIG. 7 illustrates a process 300 that may be performed by a system in accordance with present embodiments, wherein certain motion types are identified and actions are taken to limit false alarms or to facilitate identification of potential causes of false alarms. The process 300 begins with receiving measurements from at least one accelerometer, as represented by block 302. Next, as illustrated by block 304, the measurements from the accelerometer are recorded over time. Accelerometers generally function to measure acceleration minus gravitational acceleration, and, thus, an accelerometer at rest will generally indicate approximately (negative of) gravitational acceleration. Accordingly, relative measurements of the accelerometer may be utilized to identify motion of the accelerometer. As represented by block 306, the process 300 includes analyzing and/or comparing the recorded measurements provided in block 304 to identify motion. Further, block 306 may include a step for identifying certain patterns in the accelerometer measurements that are indicative of certain types of motion, as represented by block 308.

Once motion has been identified, whether it is merely motion that exceeds a particular threshold or a particular type of motion indicated by a pattern, present embodiments may perform one or more actions relative to an ECG obtained simultaneously with the analysis of the data from the accelerometer, as represented by blocks 310-316. For example, a present embodiment may generally suppress alarms based on changes in the ECG for a time period (block 310), suppress only alarms for patterns in the ECG that are associated by clinical data with the identified type of motion (block 312), modify the ECG to eliminate noise based on empirical data that correlates a specific noise value with the identified type of motion (block 314), and/or provide an indication of the motion that occurred during or proximate the time at which the alarm was initiated (block 316). With regard to block 310, a time period may be set based on the typical time required to recover from a particular type of noise or any noise. Further, the time may run from the time of the last detected motion. However, there may be a maximum amount of time allowed for suppression such that constant movement will not suppress all alarms. With regard to blocks 312 and 314 various different types of motion and/or corresponding noise values may be obtained via clinical trials and the resulting empirical data may be stored in data tables in a memory of an electrocardiograph such that patterns may be compared and identified when a substantial match is made. As a specific example, a certain motion pattern may be identified during monitoring and associated with a particular type of noise via a data table stored in memory that includes patterns and motion types that have been identified through clinical trials. Further, the motion type may be correlated by empirical data with a particular noise pattern and that noise pattern may be subtracted from the electrocardiograph signal to produce a corrected signal. The result of the method 300 may include a reduction in nuisance alarms and/or more efficient utilization of a caregiver's time.

Another aspect of present embodiments includes the use of motion detection features to provide supplemental diagnostic data. For example, one or more accelerometers (e.g., a tri-axis accelerometer) may be utilized to measure additional heart and/or lung information (e.g., heart rate, breathing rate, and lung sounds) in between motion events. Indeed, certain subtle motions may be detected that are indicative of certain heart and lung activities. For example, different directional motions or motions detected by accelerometers positioned in different locations on the patient while the patient is at rest may be indicative of particular valve movements in the heart and/or certain lung motions (e.g., breathing). These subtle motions may be detected and utilized for patient analysis. For example, certain heart motions may be indicative of congestive heart failure and certain lung motions may be indicative of lung congestion. Accordingly, the utilization of accelerometers may not only improve utilization of an associated ECG obtained during motion events but may also supplement the data provided by the ECG between motion events.

Technical effects of the invention may include facilitating the reduction and/or identification of false alarms due to motion artifact, obtaining simultaneous motion measurements for diagnostic purposes with negligible additional power requirements, identifying commonly encountered patient motion in continuous care settings to facilitate monitoring and diagnosis, detecting and identifying certain body position changes (e.g., supine and lateral), detecting motions associated with certain patient conditions, and so forth. Specifically, for example, motion artifact may be detected and utilized to suppress alarms or modify data to negate noise. As another example, motion detection may be utilized to identify heart rate, opening and closure of heart valves, lung movement, patient motion characterized during liver and lung ablation, and blood flow motion.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system, comprising:
an electrocardiograph;
a plurality of sensors communicatively coupled with the electrocardiograph, wherein each of the plurality of sensors comprises an electrode capable of detecting electrical impulses generated by a patient's body and transmitting signals indicative of detected electrical impulses to the electrocardiograph;
a motion detection feature communicatively coupled with the electrocardiograph, wherein the motion detection feature is capable of detecting movement of the patient's body and providing signals indicative of detected movement to the electrocardiograph; and
wherein the electrocardiograph is capable of detecting a particular type of patient motion and/or patient position based on the signals indicative of the detected motion, capable of providing output based on the signals indicative of the detected electrical impulses, and capable of providing output based on the signals indicative of the detected movement.

2. The system of claim 1, wherein the motion detection feature comprises an accelerometer or a gyro.

3. The system of claim 1, wherein the motion detection feature is integral with a one of the plurality of sensors.

4. The system of claim 3, wherein the motion detection feature communicatively couples with the electrocardiograph via a communication cable shared with the one of the plurality of sensors.

5. The system of claim 1, wherein the electrocardiograph is configured to detect the particular type of patient motion based on comparison of a trend of measurements obtained from the motion detection feature with empirical data.

6. The system of claim 1, wherein the electrocardiograph is configured to activate an alarm upon detection of certain electrical signals from the patient and to suppress the alarm for a period of time based on detection of motion based on measurements from the motion detection feature.

7. The system of claim 1, wherein the motion detection feature is integral with a motion detection sensor separate from the plurality of sensors.

8. The system of claim 1, wherein the motion detection feature is capable of detecting organ movement within the patient's body when the patient's body is substantially motionless and wherein the electrocardiograph is capable providing supplemental diagnostic information based on the detected organ movement.

9. The system of claim 1, comprising a plurality of motion detection features.

10. A electrocardiograph monitor, comprising:
one or more inputs capable of receiving signals from an electrode capable of detecting electrical impulses from a patient's body and signals from a motion detection feature capable of detecting movement of the patient's body;
a processor capable of identifying a type of motion and/or posture of the patient's body based on the signals from the motion detection feature; and
an alarm mechanism capable of providing an audible, tactile, or visual alert upon detection of a certain level or pattern of the electrical impulses and capable of providing a corresponding indication of the type of motion and/or posture of the patient's body based on the signals from the motion detection feature.

11. The electrocardiograph monitor of claim 10, wherein the one or more inputs is capable of receiving signals from an accelerometer and/or a tri-axis accelerometer.

12. The electrocardiograph monitor of claim 10, wherein the processor is capable of identifying various types of organ motion based on the signals from the motion detection feature when the patient's body is substantially at rest.

13. The electrocardiograph monitor of claim 12, wherein the processor is capable of identifying heart valve movement, breathing, and heart rate based on the signals from the motion detection feature when the patient's body is substantially at rest.

14. A method, comprising:
receiving measurements from a motion detection feature capable of detecting movement and receiving measurements from an electrode capable of detecting cardiac electrical impulses;
recording the measurements from the motion detection feature and the electrode;
identifying the presence of a level or type of movement based on the recorded measurements; and
suppressing an alarm generated by the measurements from the electrode based on the identified movement.

15. The method of claim 14, comprising suppressing the alarm for a designated period of time.

16. The method of claim 14, wherein identifying the presence of the level or type of movement comprises identifying a pattern in the measurements from the motion detection feature and identifying a correspondence between the pattern and a particular type of movement based on data stored in a memory.

17. The method of claim 16, comprising suppressing a particular type of alarm based on the particular type of movement while other alarms remain unsuppressed.

18. The method of claim 16, comprising providing a graphical indication of the particular type of movement, wherein the graphical indication comprises an icon or text.

19. The method of claim 16, comprising modifying an electrocardiogram to reduce noise identified as being caused by the presence of the level or type of movement.

20. The method of claim 16, wherein receiving measurements from the motion detection feature comprises receiving measurements from an accelerometer or a gyro transmitted along a communication cable that also transmits the measurements from the electrode.
